(19) United States
(12) Patent Application Publication
(10) Pub. No.: US 2026/0306170 A1
(43) Pub. Date: Oct. 8, 2026

(54) END CAP DEVICE FOR MANIPULATING TISSUE, RELATED SYSTEMS, AND RELATED METHODS OF USE

(71) Applicant: Boston Scientific Scimed, Inc., Maple Grove, MN (US)

(72) Inventors: Nathan Thomas CUMMINGS, Worcester, MA (US); Sean POWELL, Holden, MA (US)

(73) Assignee: Boston Scientific Scimed, Inc., Maple Grove, MN (US)

(21) Appl. No.: 19/632,720

(22) Filed: Mar. 30, 2026

Related U.S. Application Data
(60) Provisional application No. 63/782,105, filed on Apr. 2, 2025.

(51) Int. Cl. A61B 18/18; A61B 18/00

(52) U.S. Cl. CPC A61B 18/18; A61B 2018/00083; A61B 2018/00601; A61B 2018/00964

(57) ABSTRACT

A medical system includes a medical device and an end cap device coupled to the distal end of the shaft. The medical device includes a handle and a shaft extending distally from the handle to a distal end. The shaft includes a working channel. The end cap device includes a ring, a plurality of tubes coupled to the ring, and one or more wires movably extending through the tubes. At least a portion of the one or more wires include one or more surface features configured to engage with and lift tissue.

### CROSS-REFERENCE TO RELATED APPLICATIONS

This application claims priority to U.S. Provisional Application No. 63/782,105, filed on Apr. 2, 2025, which is incorporated by reference herein in its entirety.

### TECHNICAL FIELD

Various aspects of this disclosure relate generally to end caps for medical devices, related systems, and related methods of use. In particular, aspects of this disclosure relate to end cap devices for manipulating tissue, for example, with one or more movable wires with surface features to interact with and manipulate tissue at a treatment site.

### BACKGROUND

Medical procedures may involve removing a lesion or other tissue from a body lumen. For example, endoscopic mucosal resection (EMR) and endoscopic submucosal dissection (ESD) utilize medical instruments to remove cancerous lesions or other tissue from the gastrointestinal tract. For example, medical instruments (e.g., an electrosurgical knife or a hemostatic device) may be passed through a working channel of an endoscope to cut, puncture, perforate, or otherwise treat a lesion or tissue at a treatment site. In some instances, the lesion or tissue that has been cut, punctured, or perforated at the treatment site may interfere with additional treatments. For example, the cut tissue may impair the ability to cut, treat, or otherwise access other tissue, may impair the visualization of the tissue or treatment site by illumination and/or visualization devices of the endoscope, etc. These concerns may increase the duration, costs, and risks of the medical procedure.

The devices, systems, and methods of this disclosure may help to rectify one or more of the deficiencies described above or address other aspects of the art.

### SUMMARY

Each of the aspects disclosed herein may include one or more aspects of the features described in connection with any of the other disclosed aspects.

In one aspect, a medical system may include a medical device and an end cap device coupled to the distal end of the shaft. The medical device may include a handle and a shaft extending distally from the handle to a distal end. The shaft may include a working channel. The end cap device may include a ring, a plurality of tubes coupled to the ring, and one or more wires movably extending through the tubes. At least a portion of the one or more wires may include one or more surface features configured to engage with and lift tissue.

The medical system may include one or more of the following features. The end cap device may include one or more tracks extending from a distal end of one tube of the plurality of tubes to a distal end of another tube of the plurality of tubes. The end cap device may include four tubes, two tracks, and two wires. A first pair of tubes of the four tubes may be spaced a first angular spacing apart circumferentially around the shaft, and a second pair of tubes of the four tubes may be spaced a second angular spacing apart circumferentially around the shaft. The second angular spacing may be smaller than the first angular spacing. Each wire of the two wires may be configured to move independently of the other wire. The end cap device may include three tubes, two tracks, and two wires. The three tubes may include one shared tube, and the two wires may both extend through the shared tube. The two wires may be configured to move in tandem. The distal end of the shaft may include one or more lighting devices and one or more imaging devices. The tracks may extend in a curved shape such that the tracks are spaced away from the distal end of the shaft. The tracks may be spaced away radially from the working channel relative to a central longitudinal axis of the shaft.

The one or more surface features on the one or more wires may be biased in a first direction such that movement in the first direction causes the one or more surface features to engage with tissue, and such that movement in a second direction opposite to the first direction causes the one or more surface features to disengage with the tissue. The shaft may include an articulation portion, and the end cap device may be coupled to the shaft at a position distal to the articulation portion. The working channel may be configured to movably receive an electrosurgical knife. One or more portions of the end cap device may be formed of one or more non-conductive materials. Each of the one or more wires may be formed a plastic material.

In another aspect, an end cap device for a medical device includes a ring, a plurality of tubes coupled to the ring, a plurality of tracks, and a plurality of wires. Each track of the plurality of tracks may be coupled to distal ends of two tubes of the plurality of tubes. Each wire of the plurality of wires may be movably extendable through two of the tubes and along one of the tracks. At least a portion of each of the wires includes surface features may be configured to engage with and lift tissue.

The end cap device may include one or more of the following features. The tracks each may form a curved shape extending between the distal ends of the two tubes. The end cap device may include four tubes, two tracks, and two wires. A first pair of tubes of the four tubes may be spaced a first angular spacing apart circumferentially around the ring. A second pair of tubes of the four tubes may be spaced a second angular spacing apart circumferentially around the ring. The second angular spacing may be smaller than the first angular spacing.

In yet another aspect, a method for treating tissue may include navigating a medical system through a body lumen to a treatment site. The medical system may include a medical device, which may include a shaft. The shaft may include a working channel. The medical system may also include an end cap device coupled to a distal end of the shaft. The end cap device may include a ring, a plurality of tubes coupled to the ring, a plurality of tracks, and a plurality of wires. Each track of the plurality of tracks may be coupled to distal ends of two tubes of the plurality of tubes. Each wire of the plurality of wires may be movably extendable through two of the tubes and along one of the tracks. At least a portion of each of the wires may include surface features configured to engage with and lift tissue. The method may further include extending a medical instrument through the working channel of the shaft, cutting tissue at the treatment site with the medical instrument, and moving one or more of the wires to lift at least a portion of the cut tissue at the treatment site.

The method may further include cutting additional tissue at the treatment site with the medical instrument.

### BRIEF DESCRIPTION OF FIGURES

The accompanying drawings, which are incorporated in and constitute a part of this application, illustrate exemplary aspects of this disclosure and together with the description, serve to explain the principles of this disclosure.

FIGS. 1A and 1B illustrate an exemplary medical system including a medical device and an end cap device coupled to a distal end of the medical device, according to aspects of this disclosure.

FIG. 2 illustrates a side view of distal portions of the medical device, the end cap device, and a treatment instrument at a treatment site.

FIG. 3 is an end view of the distal end of the medical device and another exemplary end cap device.

FIG. 4 is an end view of the distal end of the medical device and yet another exemplary end cap device.

FIG. 5 depicts an exemplary flow chart for a method of treating a treatment site.

### DETAILED DESCRIPTION

Particular aspects of this disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Various figures include an arrow labeled “P” to indicate the proximal direction, and also include an arrow labeled “D” to indicate the distal direction.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although endoscopes are referenced herein for illustration purposes, it will be appreciated that this disclosure encompasses any suitable delivery device or medical device configured to allow an operator to access and view internal body anatomy of a subject (e.g., patient) or to deliver medical or treatment instruments, such as, for example, electrosurgical knives, electrodes, biopsy forceps, graspers, baskets, snares, probes, scissors, retrieval devices, lasers, and other tools, into the subject’s body. The medical devices herein may be inserted into a variety of body lumens and/or cavities, such as, for example, the urinary tract or gastrointestinal tract. It will be appreciated that, unless otherwise specified, bronchoscopes, duodenoscopes, ureteroscopes, gastroscopes, endoscopic ultrasonography (“EUS”) scopes, colonoscopes, laparoscopes, cystoscopes, aspiration scopes, sheaths, catheters, or any other suitable delivery device or medical device may be used in connection with the features described herein.

This disclosure includes end caps for medical devices. Features of the end caps herein may improve control of components involved with cutting tissue and/or stopping bleeding of tissue (e.g., tissue coagulation) by coupling movement of the components with movement of a medical device (e.g., 1-to-1 movement), among other aspects. Improved control of tissue cutting and/or tissue coagulation components may reduce procedure times and may improve efficiency and safety of medical procedures, such as EMR or ESD, among other aspects.

According to some aspects of this disclosure, the end cap may be coupled to a distal end of a medical device, while allowing other treatment instruments (e.g., an electrosurgical knife, an injection needle, a grasper, forceps, a snare, etc.) to be extended through a working channel of the medical device. The end cap may be rigidly coupled to the distal end of the medical device, which may allow for an operator to control movement of the end cap via movement of the medical device (e.g., 1-to-1 movement). For example, the distal end of the medical device may be moved (e.g., moved in a longitudinal direction, deflected, or rotated) to correspondingly move or otherwise position the end cap.

As discussed in detail below, the end cap may include and support the movement of one or more wires with one of more surface features. The wires may engage with and help to lift tissue (e.g., tissue that has been cut or is in the process of being cut) at a treatment site. As such, the one or more wires may help to separate tissue from the remainder of the treatment site. The one or more wires may also help the user access other portions of the treatment site, for example, without the tissue impairing the access, visualization, etc. of the treatment site. For example, the one or more wires may help to lift the tissue in a direction approximately perpendicular to the treatment site, which may also be perpendicular to a longitudinal axis of the medical device, working channel, one or more treatment instruments delivered through the working channel, etc.

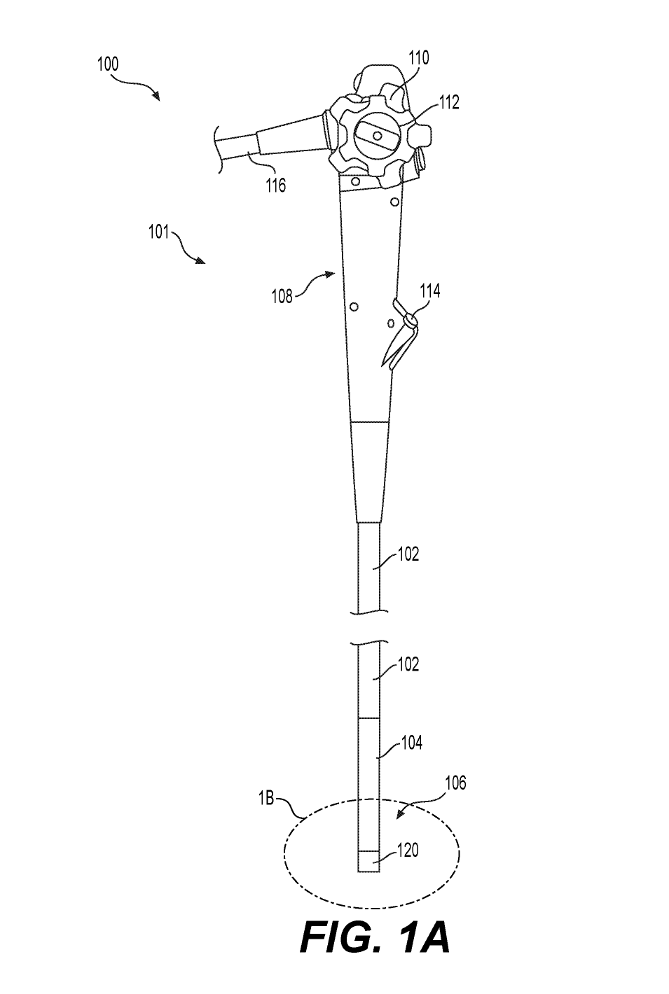

FIG. 1A illustrates an exemplary medical system 100, and FIG. 1B illustrates a distal portion of medical system 100. Medical system 100 may include a medical device 101 (e.g., an endoscope) and an end cap device 120. As discussed in detail below, end cap device 120 includes at least one ring 122 coupled to a distal portion of medical device 101. Furthermore, end cap device 120 includes one or more wires 124 that are movable relative to ring 122. Although wires 124 are discussed herein, it is noted that wires 124 may be threads, stings, filaments, sutures, etc. The one or more wires 124 include one or more barbs, ridges, spikes, textures, arms, or other surface features 126, such that the one or more wires 124 can at least partially engage with tissue at a treatment site. In these aspects and as discussed in detail below, movement of the one or more wires 124 lifts or otherwise manipulates the tissue, for example, to help separate planes or portions of tissue. End cap device 120 also includes a plurality of tubes 128, for example, connected via one or more tracks 130. As discussed in detail below, wire(s) 124 are movable through tubes 128, and track(s) 130 may help guide wire(s) 124, for example, when portions of wire(s) 124 are moving or otherwise positioned between tubes 128. In these aspects, track(s) 130 may help support portions of wire(s) 124, for example portions of wire(s) that are distal to medical device 101.

Referring to FIG. 1A, medical device 101 may include a handle 108 and a shaft 102, with shaft 102 extending distally from handle 108. Shaft 102 may include an articulation section 104 and a distal tip or end 106. As shown in FIG. 1B, a distalmost face 162 of shaft 102 may include an imaging device 180 (e.g., a camera, an imager, etc.), one or more light sources 182 (e.g., light emitting diode(s), optical fibers, etc.), and a distal opening in communication with a working channel 118 extending through shaft 102. Referring back to FIG. 1A, handle 108 may include a port 114 in fluid communication with working channel 118. Handle 108 may include one or more actuators, for example, a first actuator 110 and a second actuator 112 (e.g., knobs or levers), for controlling articulation section 104 of shaft 102. For example, actuators 110, 112 may control movement of articulation section 104 of shaft 102 in multiple directions, for example, movement along different planes. Handle 108 may be coupled to an umbilicus 116. Umbilicus 116 may extend from handle 108 to capital equipment, such as electronic controller(s), one or more displays, one or more user interfaces, and/or source(s) of air, water, and/or suction.

End cap device 120 may be secured to distal end 106 of shaft 102 via a friction fit or other coupling mechanism. As shown in FIG. 1B, end cap device 120 may include ring 122, and ring 122 may be coupled to an outer surface of distal end 106 of shaft 102, for example, proximal of distalmost face 162 of shaft 102. In some aspects, ring 122 is coupled to shaft 102 at a position distal to articulation section 104. Ring 122 may be generally cylindrical. In some aspects, ring 122 may be adjustable, for example, to form different sized circumferences or different shapes to be coupled to different sizes or shapes of scopes.

End cap device 120 may include a plurality of tubes 128, and one or more wires 124 may movably extend through respective lumens 128A of each of tubes 128. It is noted that tubes 128 may be adjacent to the distal end of shaft 102. For example, a distal end of each tube 128 may be adjacent to the distal end of shaft 102. In other examples, the distal end of one or more tubes 128 may extend distal to the distal end of shaft 102, for example, distal to distalmost face 162.

Furthermore, end cap device 120 may include one or more tracks 130 or support elements. For example, as mentioned above, one track 130 may extend from a distal end of one tube 128 to a distal end of another tube 128. Each track 130 may extend in a partially circular or otherwise curved configuration between the distal ends of the respective tubes 128. Tracks 130 may include a groove or indented portion 130A, which may receive a distal portion 124D (e.g., the portion extended or otherwise exposed from tubes 128) of wire 124. Indented portion 130A may help to support or guide one wire 124 as wire 124 moves between tubes 128. In these aspects, tracks 130 may help to support or guide the movement of wires 124 relative to end cap device 120 and distal end 106 of shaft 102 (e.g., to be spaced away from distalmost face 162 of shaft 102). Additionally, the positioning of tubes 128 and tracks 130 may help to ensure that end cap device 120 (e.g., including wires 124 and tracks 130) does not interfere with the visualization (e.g., via imaging device(s) 180 and/or light source(s) 182) or treatment (e.g., via one or more instruments delivered through working channel 118) of medical device 101.

As shown in FIG. 1B, end cap device 120 may include four tubes 128. Relative to the orientation shown in FIG. 1B, that is, with working channel 118 arranged in a bottom (down in FIG. 1B) of medical system 100, end cap device 120 may include two bottom tubes 128B and two top tubes 128T (in the up direction in FIG. 1B). One pair of tubes, for example, bottom tubes 128B, may be spaced apart by an angular spacing A (e.g., circumferentially around shaft 102), and another pair of tubes, for example, top tubes 128T, may be spaced apart by an angular spacing B (e.g., circumferentially around shaft 102). Angular spacing B may be smaller than angular spacing A, such that top tubes 128T are closer to each other than bottom tubes 128B are to each other. For example, angular spacing A may be approximately 70 to approximately 120 degrees, for example, approximately 90 degrees, and angular spacing B may be approximately 30 degrees to approximately 90 degrees, for example, approximately 45 degrees. In some aspects, the positioning of tubes 128 on ring 122, and thus relative to other tubes 128, may be adjustable, for example, to suit a particular medical device 101, a particular procedure, etc. Additionally, tubes 128 may be positioned on the sides of distalmost face 162, which may help to ensure that wires 124 and/or tracks 130 do not impair or otherwise interfere with working channel 118, imaging device(s) 180, light source(s) 182, etc.

Additionally, bottom tubes 128B may be offset or circumferentially spaced apart from a bottom portion 102B of shaft 102, with bottom portion 102B of shaft 102 being the portion of shaft 102 below working channel 118 in FIG. 1B (e.g., a six o’clock position of shaft 102). In these aspects, bottom portion 102B of shaft 102 may be positioned in contact, adjacent to, or otherwise in proximity to tissue or other portions of a treatment site. Furthermore, the positioning of bottom tubes 128B (e.g., spaced away the bottom portion of shaft 102) may help to prevent wires 124 from inadvertently contacting tissue or other portions of the treatment site. Instead, wires 124 may contact tissue that has been cut, as discussed below.

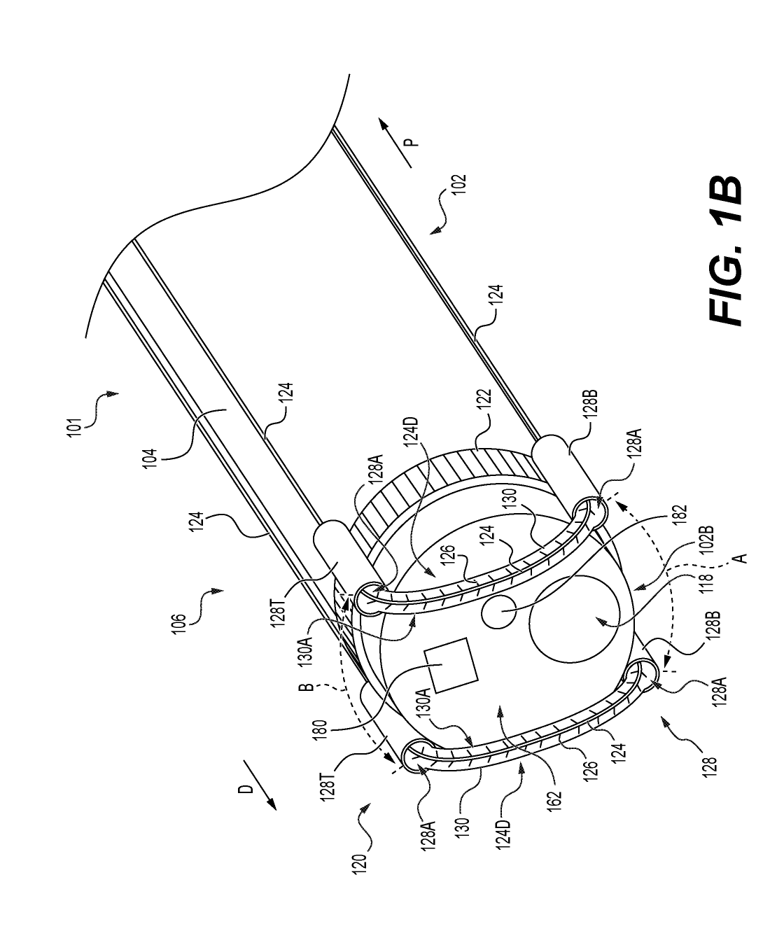

Wires 124 extend proximally, for example, to a control device (not shown). Although not shown, the control device may be integrated into or otherwise coupled to handle 108 of medical device 101. In other aspects, the control device may be separate from handle 108. Furthermore, wires 124 may extend proximally along respective circumferential portions of shaft 102 (for example, outer circumferential portions of shaft 120). Although not shown, in some aspects, shaft 102 may include one or more grooves or indentations to movably receive portions of wires 124. Alternatively or additionally, as shown in FIG. 2, medical system 100 may include one or more outer sleeves or sheaths 132 (e.g., an insertion sheath) radially surrounding other portions of medical system 100. For example, in this aspect, the outer sleeve(s) or sheath(s) 132 may radially surround portions of shaft 102 and wires 124. In other aspects, one or more of tubes 128 may extend proximally along shaft 102, for example, to a handle (e.g., handle 108 or a separate handle).

As mentioned above, wires 124 include one or more surface features 126. In some aspects, only one or more portions (e.g., distal portions 124D) of wires 124 may include surface features 126. Stated another way, proximal portions (not shown) of wires 124 (e.g., portions that extend proximally along or adjacent to shaft 102) may not include surface features 126. Additionally, in some aspects, surface features 126 are oriented, biased, or otherwise positioned on respective wires 124, such that movement of wire(s) 124 in a first direction, for example, from a bottom tube 128B toward a top tube 128T, causes surface features 126 to engage tissue and draw, pull, or otherwise manipulate the engage tissue upward (e.g., toward the respective top tube 128T). For example, surface features 126 may be angled or point at least partially upward (e.g., relative to the orientation in FIGS. 1B and 2). Furthermore, in these aspects, movement of wire(s) 124 in a second direction opposite to the first direction, for example, from a top tube 128T toward a bottom tube 128B, causes surface features 126 to disengage the tissue. In some aspects, disengaging surface feature(s) 126 of wire(s) 124 allows for the distal end of medical system 100 (e.g., distal end 106 of shaft 102) to be repositioned relative to the tissue or other portions of the treatment site.

In some aspects, medical system 100 includes two wires 124. Each of wires 124 may be independently movable. For example, one of wires 124 may be moved toward the respective top tube 128T to lift tissue adjacent to that one wire 124, while the other wire 124 is stationary. In some aspects, wires 124 may be alternatingly moved. Alternatively, in some aspects, wires 124 may move together, for example, such that manipulation of one or more proximal control devices moves both wires 124 in tandem (e.g., either up to engage with and lift tissue, or down to disengage with the tissue).

In some aspects, one or more portions of end cap device 120 may be formed of one or more non-conductive materials. For example, one or more of wires 124 (e.g., including surface features 126) may be formed of a plastic material. One or more other portions of end cap device 120 (e.g., ring 122, tubes 128, and tracks 130) may also be formed of a same or a different plastic material.

FIG. 2 illustrates a side view of distal portions of medical system 100, including distal end 106 of shaft 102 of medical device 101 and end cap device 120, positioned at a treatment site 200. As shown, end cap device 120 includes ring 122 and tubes 128 with lumens 128A. Additionally, one or more wires 124 movably extend between tubes 128, for example, at least partially supported by one or more tracks 130. Moreover, at least a portion of wire 124 (e.g., distal portion 124D) includes one or more surface features 126. It is noted that FIG. 2 illustrates two tubes 128 and one wire 124 for clarity. In some aspects, medical system 100 may include sheath 132, which may enclose or otherwise surround portions of shaft 102 and wire(s) 124. Furthermore, medical system 100 may receive one or more treatment instruments 140, for example, through working channel 118. As mentioned above, working channel 118 is in communication with port 114 (FIG. 1A). As such, one or more treatment instruments 140 may be inserted through port 114 and through working channel 118 to be delivered to treatment site 200. Treatment instrument 140 may include electrosurgical knife 142, and may be extended from working channel 118 to apply energy and/or deliver fluid to treatment site 200, for example, to help cut, separate, or otherwise treat tissue 202 (“cut tissue 202”). In some aspects, electrosurgical knife 142 may be connected to one or more fluid sources and include at least one fluid lumen, such that electrosurgical knife may be used to inject fluid into tissue (e.g., between or underneath layers of tissue).

As shown in FIG. 2, at least a portion of cut tissue 202 may at least partially engage with one or more surface features 126 of one or more wires 124 of end cap device 120. One or more wires 124 may then be manipulated to lift cut tissue 202, for example, to help separate planes or portions of tissue (e.g., cut tissue 202 from treatment site 200). For example, a first end of one or more wires 124 may be manipulated (e.g., pulled proximally) to move wire(s) 124 in a first direction E, such that at least the distal portion of wire(s) 124 move in a counterclockwise or upward direction relative to the orientation shown in FIG. 2. Wire(s) 124 may move through respective lumens 128A of tubes 128, and wire(s) 124 may be guided by one or more tracks 130. As shown, tracks 130 may help retain wires 124 in positions spaced away from distalmost face 162 of shaft 102, that is, spaced away from the distal opening of working channel 118, imaging device(s) 180, and light source(s) 182. Tracks 130 may also be spaced away (e.g., radially) from a central longitudinal axis of shaft 102. The engagement of surface features 126 with cut tissue 202 and the movement of wire(s) 124 may help to lift cut tissue 202, for example, in approximately a 90 degree direction (e.g., a 90 degree traction vector) relative to a longitudinal axis of distal end 106 of shaft 102 and/or the rest of the treatment site. In some aspects, the position of wire(s) 124 may be locked or otherwise secured, for example, to help form a constant traction vector on cut tissue 202.

In these aspects, cut tissue 202 may not interfere with the visualization of treatment site 200 (e.g., via imaging device(s) 180 and light source(s) 182). Additionally, lifting cut tissue 202 with wire(s) 124 may help to further treat treatment site 200, for example, by helping to separate cut tissue 202 from the remainder of treatment site 200. For example, treatment instrument 140 may be further extended or otherwise positioned relative to cut tissue 202 and the remainder of treatment site 200 to cut additional tissue or otherwise deliver treatment (e.g., cut, cauterize, inject fluid, etc.).

Furthermore, as mentioned above, the movement of wire(s) 124 may be reversed. For example, a second end of one or more wires 124 may be manipulated (e.g., pulled proximally) to move wire(s) 124 in a second direction F, such that at least a portion of wire(s) 124 (e.g., distal portion 124D) moves in a clockwise or downward direction relative to the orientation shown in FIG. 2. This movement may help wire(s) 124 and respective surface feature(s) 126 to disengage from cut tissue 202. In these aspects, after disengaging wire(s) 124 from cut tissue 202, one or more portions of medical system 100 may be repositioned relative to treatment site 200. Medical system 100 may repeat these steps as many times as necessary to treat treatment site 200.

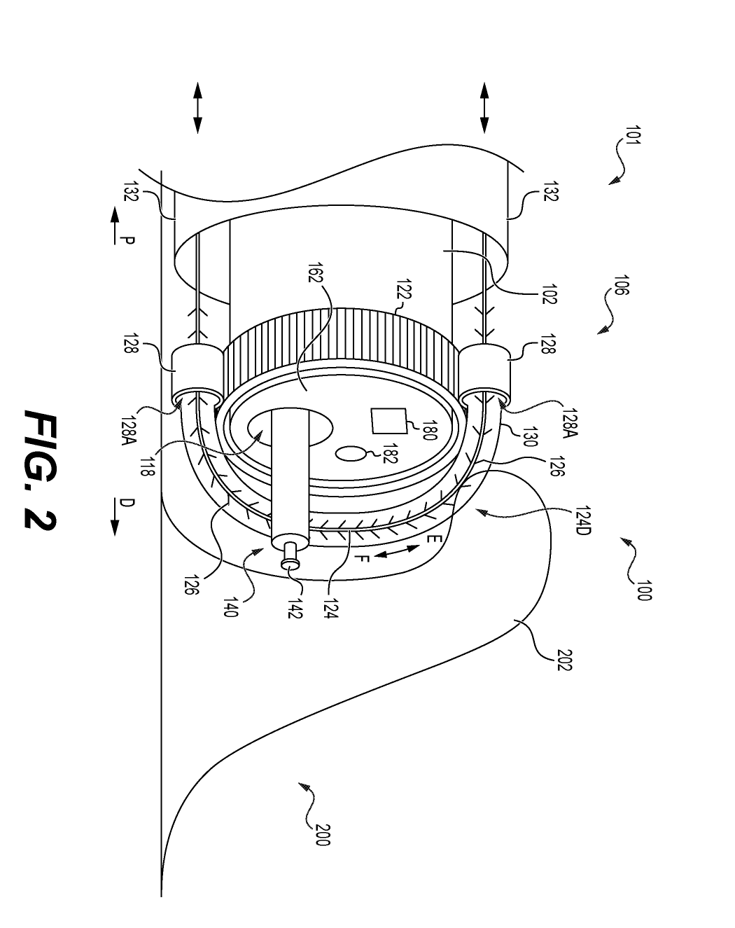

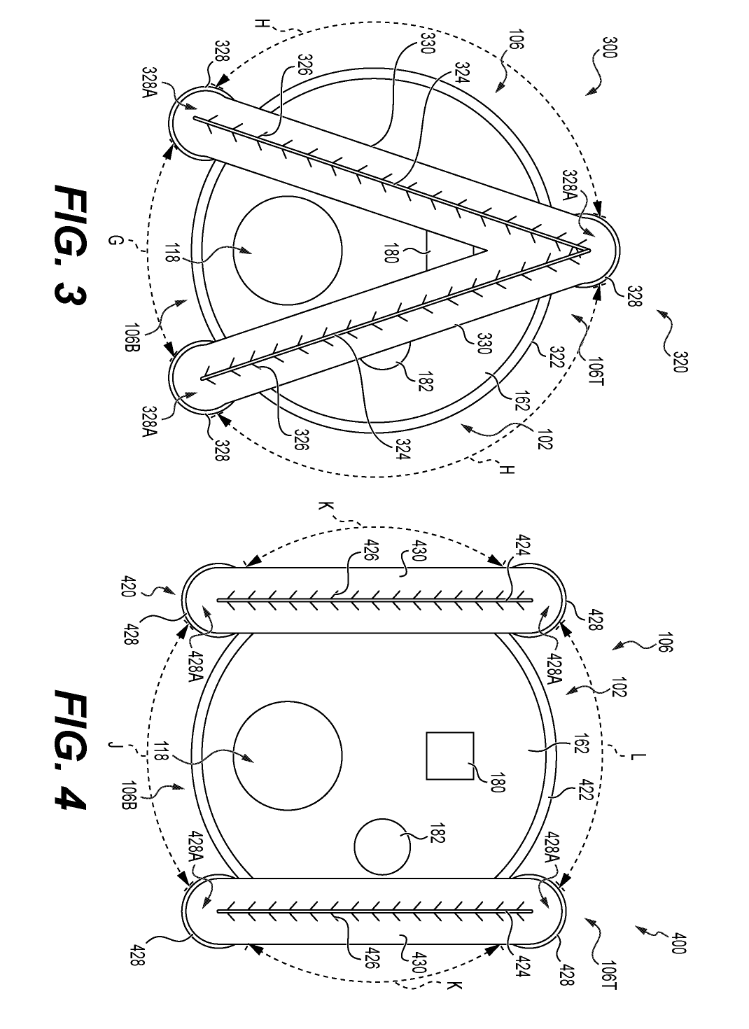

FIGS. 3 and 4 illustrate end views of alternate medical systems 300 and 400, respectively. Medical systems 300 and 400 are similar to medical system 100, except as described below. Furthermore, as shown, medical systems 300 and 400 may each include medical device 101 (FIGS. 1A and 1B), including distal end 106 of shaft 102 with a distalmost face 162. Alternatively, although not shown, medical systems 300 and 400 may include other medical devices or shafts.

As shown in FIG. 3, medical system 300 includes an end cap device 320 coupled to distal end 106 of shaft 102. End cap device 320 includes a ring 322 and wires 324, with wires 324 including surface features 326. Additionally, end cap device 320 includes a plurality of tubes 328 coupled via ring 322, with wires 324 being movable through lumens of respective tubes 328. End cap device 320 also includes tracks 330, with each track coupling distal ends of two tubes 328.

End cap device 320 includes three tubes 328, for example, with two tubes 328 at a bottom portion 106B of distal end 106 (e.g., similar to tubes 128B in FIG. 1B) and one tube 328 at a top portion 106T of distal end 106 (e.g., similar to tubes 128T in FIG. 1B). For example, tubes 328 may be spaced apart circumferentially around end cap device 320. In some aspects, tubes 328 may be evenly or unevenly spaced apart circumferentially around end cap device 320. For example, in some aspects, tubes 328 are spaced approximately 120 degrees apart circumferentially around end cap device 320. In other aspects, tubes 328 at bottom portion 106B of distal end 106 may be closer to each other (e.g., by an angle G) than a spacing between one of tubes 328 at bottom portion 106B and tube 328 at top portion 106T of distal end 106 (e.g., by an angle H). As discussed above, the tubes 328 on bottom portion 106B may be offset or circumferentially spaced apart from the bottom (e.g., relative to the orientation shown in FIG. 3) of shaft 102, such that the tubes 328 on bottom portion 106B are not directly below working channel 118. In these aspects, end cap device 320 includes two tracks 330, with both tracks 330 being coupled to the distal end of the one tube 328 at top portion 106T of distal end 106.

Furthermore, end cap device 320 includes two wires 324. One wire 324 movably extends through each of tubes 328 at bottom portion 106B of distal end 106, but both wires 324 movably extend through tube 328 at top portion 106T of distal end 106. In these aspects, tube 328 at top portion 106T of distal end 106 may be a shared tube. Wires 324 may move in two directions, as discussed above, for example, to engage with and disengage from cut tissue at a treatment site. In some aspects, wires 324 are configured to move in tandem, such that both wires move in the same direction(s) and at the same time.

As shown in FIG. 4, medical system 400 includes an end cap device 420 coupled to distal end 106 of shaft 102. End cap device 420 includes a ring 422 and wires 424, with wires 424 including surface features 426. Additionally, end cap device 420 includes a plurality of tubes 428 coupled via ring 422, with wires 424 being movable through lumens of respective tubes 428. End cap device 420 also includes tracks 430, with each track coupling distal ends of two tubes 428.

End cap device 420 includes four tubes 428, for example, with two tubes 428 at bottom portion 106B of distal end 106 (e.g., similar to bottom tubes 128B in FIG. 1B) and two tubes 428 at top portion 106T of distal end 106 (e.g., similar to top tubes 128T in FIG. 1B). For example, tubes 428 may be evenly spaced apart (e.g., by approximately 90 degrees) circumferentially around end cap device 320. In other aspects, one or more of tubes 428 may be unevenly spaced apart circumferentially around end cap device relative to one or more other tubes 428. In any of these aspects, end cap device 420 includes two tracks 430, with each track 430 being coupled to the distal end of one tube 428 at bottom portion 106B and to the distal end of one tube 428 at top portion 106T of distal end 106. In some aspects, tubes 428 at bottom portion 106B of distal end 106 may be closer to each other (e.g., by an angle J) than a spacing between one of tubes 428 at bottom portion 106B and one of tubes 428 at top portion 106T of distal end 106 (e.g., by an angle K). Additionally, tubes 428 at top portion 106T of distal end 106 may be spaced apart by an angle L. In some aspects, angle L is approximately the same as angle J. Alternatively, in other aspects, angle L is smaller than angle J. In further aspects, angle L is larger than angle J. As discussed above, the tubes 428 on bottom portion 106B may be offset or circumferentially spaced apart from the bottom (e.g., relative to the orientation shown in FIG. 3) of shaft 102, such that the tubes 428 on bottom portion 106B are not directly below working channel 118.

Furthermore, end cap device 420 includes two wires 424. One wire 424 movably extends through each tube 428 at bottom portion 106B of distal end 106, and one wire 424 movably extends through each tube 428 at top portion 106T of distal end 106. Wires 424 may move in two directions, as discussed above, for example, to engage with and disengage from cut tissue at a treatment site.

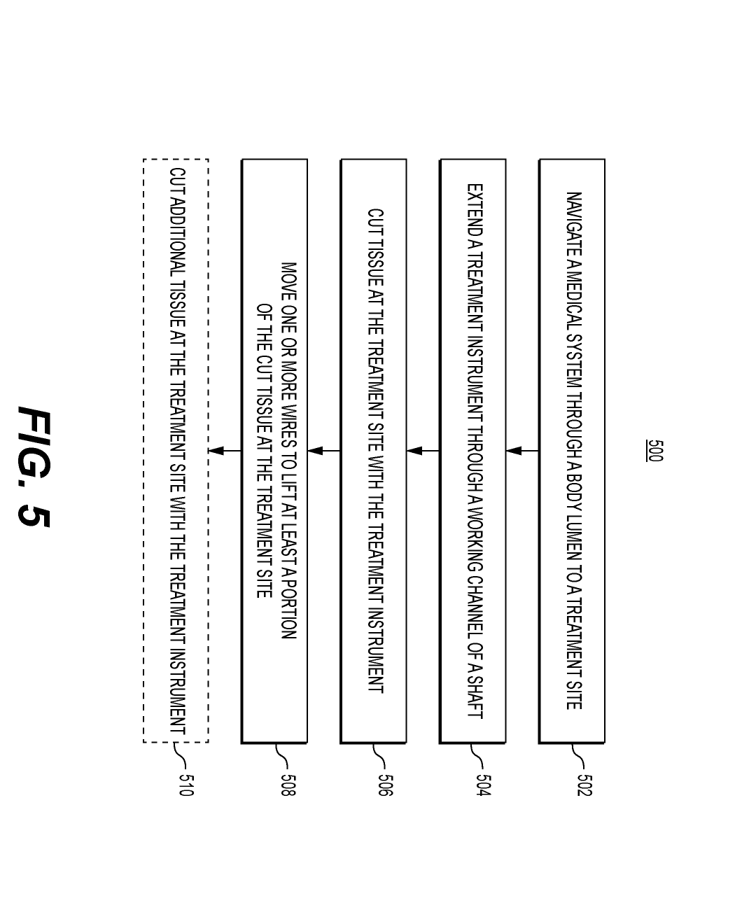

FIG. 5 depicts a method 500 of treating tissue at a treatment site with one or more of the medical systems discussed above. Method 500 includes a step 502 that includes navigating a medical system through a body lumen to a treatment site. As discussed above, the medical system (e.g., medical system 100, 300, 400) includes a medical device with a shaft, and the shaft includes a working channel. The medical system also includes an end cap device coupled to a distal end of the shaft. The end cap device includes a ring, a plurality of tubes coupled to the ring, a plurality of tracks, and a plurality of wires. Each track of the plurality of tracks is coupled to distal ends of two tubes. Each wire of the plurality of wires is movably extendable through two of the tubes and along one of the tracks. At least a portion of each of the wires includes one or more surface features configured to engage with and lift tissue. Although not shown in FIG. 5, method 500 may include a preliminary step that includes coupling the end cap device to the shaft of the medical device, for example, via a friction fit or other coupling mechanism.

Next, method 500 includes a step 504 that includes extending a treatment instrument through the working channel of the shaft. In some aspects, the medical device includes a handle with a port. The port is connected to the working channel of the shaft, and step 504 includes delivering at least a distal portion of the treatment instrument through the port and the working channel. Step 504 may include extending the distal end of the treatment instrument distally of the shaft of the medical device. In some aspects, the treatment instrument may be an electrosurgical knife or another sharp and/or energy delivery device.

Method 500 further includes a step 506 that includes cutting tissue at the treatment site with the treatment instrument. As mentioned, the treatment instrument may be an electrosurgical knife or another sharp and/or energy delivery device. Step 506 may include energizing the electrosurgical knife or other energy delivery device (e.g., an electrode). Step 506 may include positioning a distal end of the treatment instrument between layers of tissue at the treatment site and energizing the treatment instrument and/or delivering one or more fluids to help separate layers of tissue. Additionally, one or more portions or components of the medical device (e.g., imaging device 180 and one or more light sources 182 at distal end 106 of shaft 102) may assist with visualizing the treatment instrument and the tissue at the treatment site throughout the procedure.

Next, method 500 includes a step 508 that includes moving one or more wires of the end cap device to lift at least a portion of the cut tissue at the treatment site. As discussed above, moving the one or more wires (e.g., in the counterclockwise direction shown in FIG. 2) may lift the cut tissue at an approximately 90 degree angle from the treatment site and/or the longitudinal axis of the shaft of the medical device. Lifting at least a portion of the cut tissue may help the user visualize (e.g., with imaging device 180 and one or more light sources 182) the treatment site, for example, to evaluate the success and/or progress of the cutting of the tissue or other treatments being delivered. Furthermore, lifting at least a portion of the cut tissue may help the user access any remaining tissue, for example, to complete the cutting, apply cauterization energy, inject fluid(s), etc.

In some aspects, method 500 may include an optional step 510 that includes cutting additional tissue at the treatment site with the treatment instrument. For example, with the cut tissue lifted via the one or more wires, the user may extend the treatment instrument further distally to cut additional tissue at the treatment site. Alternatively or additionally, the treatment instrument (or another medical instrument) may be used to deliver one or more other treatments (e.g., delivering fluid, delivering or applying a coagulant, delivering cauterization energy, etc.). Additionally, although not shown, in some aspects, method 500 may include moving one or more wires in a second, opposite direction (e.g., clockwise in FIG. 2), for example, to disengage the wires and surface features from tissue. The method may then include repositioning one or more components of the medical system relative to the cut tissue, treatment site, etc.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

1. A medical system comprising:
a medical device comprising:
a handle; and
a shaft extending distally from the handle to a distal end, wherein the shaft includes a working channel; and
an end cap device coupled to the distal end of the shaft, the end cap device comprising:
a ring;
a plurality of tubes coupled to the ring; and
one or more wires movably extending through the tubes, wherein at least a portion of the one or more wires include one or more surface features configured to engage with and lift tissue.

2. The medical system of claim 1, wherein the end cap device includes one or more tracks extending from a distal end of one tube of the plurality of tubes to a distal end of another tube of the plurality of tubes.

3. The medical system of claim 2, wherein the end cap device includes four tubes, two tracks, and two wires.

4. The medical system of claim 3, wherein a first pair of tubes of the four tubes are spaced a first angular spacing apart circumferentially around the shaft, wherein a second pair of tubes of the four tubes are spaced a second angular spacing apart circumferentially around the shaft, and wherein the second angular spacing is smaller than the first angular spacing.

5. The medical system of claim 3, wherein each wire of the two wires is configured to move independently of the other wire.

6. The medical system of claim 2, wherein the end cap device includes three tubes, two tracks, and two wires.

7. The medical system of claim 6, wherein the three tubes include one shared tube, and wherein the two wires both extend through the shared tube.

8. The medical system of claim 7, wherein the two wires are configured to move in tandem.

9. The medical system of claim 2, wherein the distal end of the shaft includes one or more lighting devices and one or more imaging devices, and wherein the tracks extend in a curved shape such that the tracks are spaced away from the distal end of the shaft.

10. The medical system of claim 9, wherein the tracks are spaced away radially from the working channel relative to a central longitudinal axis of the shaft.

11. The medical system of claim 1, wherein the one or more surface features on the one or more wires are biased in a first direction such that movement in the first direction causes the one or more surface features to engage with tissue, and such that movement in a second direction opposite to the first direction causes the one or more surface features to disengage with the tissue.

12. The medical system of claim 1, wherein the shaft includes an articulation portion, and wherein the end cap device is coupled to the shaft at a position distal to the articulation portion.

13. The medical system of claim 1, wherein the working channel is configured to movably receive an electrosurgical knife.

14. The medical system of claim 1, wherein one or more portions of the end cap device are formed of one or more non-conductive materials.

15. The medical system of claim 1, wherein each of the one or more wires is formed a plastic material.

16. An end cap device for a medical device, the end cap device comprising:
a ring;
a plurality of tubes coupled to the ring;
a plurality of tracks, wherein each track of the plurality of tracks is coupled to distal ends of two tubes of the plurality of tubes; and
a plurality of wires, wherein each wire of the plurality of wires is movably extendable through two of the tubes and along one of the tracks, and wherein at least a portion of each of the wires includes surface features configured to engage with and lift tissue.

17. The end cap device of claim 16, wherein the tracks each form a curved shape extending between the distal ends of the two tubes.

18. The end cap device of claim 16, wherein the end cap device includes four tubes, two tracks, and two wires, wherein a first pair of tubes of the four tubes are spaced a first angular spacing apart circumferentially around the ring, wherein a second pair of tubes of the four tubes are spaced a second angular spacing apart circumferentially around the ring, and wherein the second angular spacing is smaller than the first angular spacing.

19. A method for treating tissue, the method comprising:
navigating a medical system through a body lumen to a treatment site, the medical system comprising:
a medical device comprising a shaft, wherein the shaft includes a working channel; and
an end cap device coupled to a distal end of the shaft, wherein the end cap device includes:
a ring;
a plurality of tubes coupled to the ring;
a plurality of tracks, wherein each track of the plurality of tracks is coupled to distal ends of two tubes of the plurality of tubes; and
a plurality of wires, wherein each wire of the plurality of wires is movably extendable through two of the tubes and along one of the tracks, and wherein at least a portion of each of the wires includes surface features configured to engage with and lift tissue;
extending a medical instrument through the working channel of the shaft;
cutting tissue at the treatment site with the medical instrument; and
moving one or more of the wires to lift at least a portion of the cut tissue at the treatment site.

20. The method of claim 19, further comprising:
cutting additional tissue at the treatment site with the medical instrument.
